(19) United States
(12) Patent Application Publication
(10) Pub. No.: US 2025/0248977 A1
(43) Pub. Date: Aug. 7, 2025

(54) Tablet Formulation Of Tafamidis Or Pharmaceutically Acceptable Salt Thereof

(71) Applicant: Navinta LLC, Ewing, NJ (US)

(72) Inventors: Mahendra R. Patel, Delray Beach, FL (US); Niyaz Sultanbhai Mansuri, Juhapura (IN)

(21) Appl. No.: 19/042,930

(22) Filed: Jan. 31, 2025

Related U.S. Application Data
(60) Provisional application No. 63/627,884, filed on Feb. 1, 2024.
(60) Provisional application No. 63/627,886, filed on Feb. 1, 2024.

(51) Int. Cl. A61K 31/423; A61K 9/20

(52) U.S. Cl. CPC A61K 31/423; A61K 9/2018; A61K 9/205

(57) ABSTRACT

The present invention provides a pharmaceutically acceptable tablet formulation of tafamidis or a pharmaceutically acceptable salt(s) thereof or tafamidis co-crystal(s). Particularly, the present invention provides a tablet formulation of tafamidis or a pharmaceutically acceptable salt(s) thereof or tafamidis co-crystal(s) that uses water-soluble excipients and standard manufacturing process and provides an improved dissolution profile.

### FIELD OF THE INVENTION

The present invention relates to a pharmaceutically acceptable solid oral formulation of tafamidis or a pharmaceutically acceptable salt(s) thereof or tafamidis co-crystal(s). Particularly, the present invention relates to economically feasible and commercially viable pharmaceutically acceptable formulations of tafamidis or a pharmaceutically acceptable salt(s) thereof or tafamidis co-crystal(s) prepared with water soluble excipients. More particularly, the present invention relates to a tablet formulation of tafamidis or a pharmaceutically acceptable salt(s) thereof or tafamidis co-crystal(s) that uses water-soluble excipients and standard manufacturing process, wherein the formulation provides a faster dissolution profile.

### BACKGROUND OF THE INVENTION

Tafamidis (Brand: VYNDAMAX®) and Meglumine salt of Tafamidis (Brand: VYNDAQEL®) in soft gelatin capsule dosage form have been approved by USFDA in 2019 for the treatment of the cardiomyopathy of wild type or hereditary transthyretin-mediated amyloidosis in adults. Chemically, Tafamidis is known as 2-(3,5-dichlorophenyl)-1,3-benzoxazole-6-carboxylic acid and Tafamidis meglumine is known as 2-(3,5-Dichlorophenyl)-1,3-benzoxazole-6-carboxylic acid-1-deoxy-1-(methyl-amino)-D-glucitol. The structures of Tafamidis (Formula-I) and Tafamidis meglumine (Formula-II) are as follows:

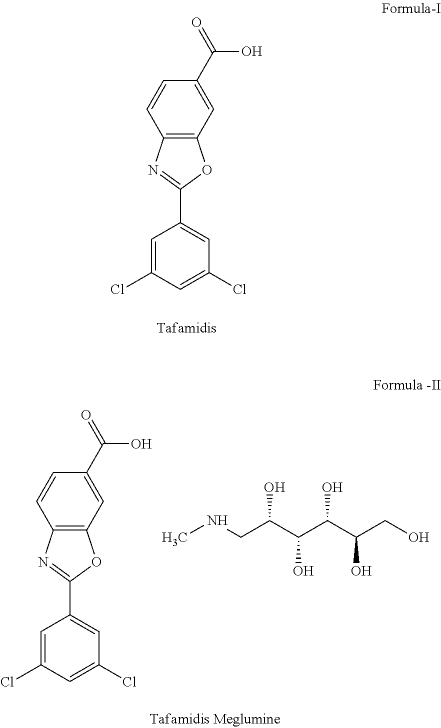 [SMILES: O=C(O)c1ccc2nc(-c3cc(Cl)cc(Cl)c3)oc2c1 ; O=C(O)c1ccc2nc(-c3cc(Cl)cc(Cl)c3)oc2c1 ; CNC[C@H](O)[C@@H](O)[C@H](O)[C@H](O)CO]

Tafamidis capsules 61 mg (VYNDAMAX®, Pfizer) is approved and available as a soft gelatin capsule. It is administered through oral route for the treatment of cardiomyopathy.

The active pharmaceutical ingredient (API) Tafamidis has a low aqueous solubility (BCS class-IV compound) and is provided in the form of suspended particles in an oily vehicle in the approved soft gelatin formulation.

Tafamidis meglumine capsules 20 mg (Vyndaqel®, Pfizer) is approved and is also available as a soft gelatin capsule. It is administered through oral route for the treatment of cardiomyopathy. Formulation of Tafamidis meglumine capsule also contains similar excipients as Tafamidis formulation and utilizes complex process to manufacture soft gelatin capsules. According to the VYNDAQEL® European Public assessment report, “during development it was shown that tafamidis active substance, when formulated in a hard gelatin capsule with standard excipients, formed a rigid gel structure upon contact with aqueous vehicles thereby affecting the dissolution rate from the solid dosage.” The VYNDAQEL® European Public assessment report further states that “the chosen excipients in the capsule fill solution are: polyethylene glycol (suspending vehicle), sorbitan monoleate (surfactant/wetting agent), polysorbate 80 (surfactant/wetting agent),” while “the excipients in the capsule shell are: gelatin, propylene glycol, purified water, ‘Sorbitol special-glycerin blend’ (d-sorbitol, 1,4 sorbitan, mannitol and glycerin) and titanium dioxide (E 171) and black printing ink.”

The available soft gelatin formulation is associated with critical process requirements and requires specialized equipment set up. The soft gelatin formulation is relatively complex as compared to powder filled hard gelatin capsule dosage form. It is less preferred due to its complexity, manufacturing process/steps and equipment requirements. The manufacturing process for the soft gelatin capsule generally consists of mixing, homogenizing, de-aerating, encapsulation, drying, washing, in-line printing and packaging.

Dissolution performance for the reference listed drug (RLD) soft gelatin capsule (VYNDAMAX®) was identified as a major formulation hurdle (i.e., slow dissolution), for which a suspension-filled capsule was targeted for development. For the liquid suspension, a complex formulation was used, which included a base excipient that readily dispersed in aqueous media, a surfactant to promote dispersion and dissolution of the active substance in the formulation, a viscosity enhancing agent to promote suspension homogeneity during manufacture, and an antioxidant to minimize the risk of oxidative degradation and gelatin cross-linking. PEG 400 was used as the water miscible liquid base to promote rapid dispersion of Tafamidis suspension, following mixing with aqueous media during dissolution. Polysorbate 20 was selected as the surfactant based on the degree to which it is miscible with PEG 400 and able to maintain a homogeneous bulk fill. In combination, PEG 400 and polysorbate 20 constitute a base to which other excipients were added and assessed separately in prototypes manufactured exclusively for stability. Povidone K-90 was included to enhance viscosity in PEG-based liquids and reduce settling during manufacture. The soft gelatin capsule formulation available on the market is sensitive to higher temperature storage and results in significant yield loss due to cumbersome processing requirements.

U.S. Pat. No. 11,523,993 discloses dosage forms of tafamidis and its pharmaceutically acceptable salt, including both tablet and hard gelatin capsule compositions. The tablet composition comprises tafamidis or its salt, at least one acidifier, and at least one pharmaceutically acceptable excipient. The hard gelatin capsule composition comprises spray dried or evaporated composition comprising at least one solubility enhancer, at least one disintegrant and at least one pharmaceutically acceptable excipient. The patent discusses the issues of formation of rigid gels in powder filled hard capsule formulations of tafamidis and tafamidis meglumine and states that the presence of an acidifier in the hard gel capsule formulation is necessary to inhibit gel formation during the dissolution. The patent further emphasizes the use of granulation and spray drying or evaporating methods, which are complex, time consuming and comparatively expensive.

US Pat. Pub. No. 2022/0370365 discloses powdered formulations of tafamidis, wherein the formulations contain a carrier selected from magnesium aluminometasilicate, microcrystalline cellulose, low substituted hydroxypropyl cellulose, crospovidone and calcium silicate. The publication discusses disadvantages of soft gel capsule formulations of tafamidis and states that the disclosed invention overcomes these disadvantages by providing a powdered preparation that is optimal for tablet preparation. The publication further states that using the specifically disclosed carriers as adsorption carriers is necessary to suppress the decrease in the elution rate of tafamidis from the powdered preparation to achieve the objective of the disclosed invention.

WO 2024/084362 discloses soft gel capsule formulations containing crystalline forms of Tafamidis, in particular Tafamidis Form 6, and methods for their preparation. The publication describes solubility problems with soft gel capsule formulations of Tafamidis and the objective of providing a soft gel capsule formulation of Tafamidis that is stable and is prepared by the specifically disclosed methods, i.e., the process of granulation using fluid bed granulator (top-spray granulation), which is complex and time consuming.

U.S. Pat. Pub. No. 2023/0149365 discloses hard gelatin or HPMC capsule or tablet formulations of Tafamidis or Tafamidis meglumine that do not form rigid gels upon contacting with water or buffer solution. The publication describes hard gelatin or HPMC capsule formulations having easily dispersible granules and comprising at least one acidifier, as well as hard gelatin or HPMC capsule formulations having a spray-dried or evaporated composition.

U.S. Pat. No. 11,795,152 discloses crystalline forms of tafamidis such as Tafamidis fumaric acid co-crystal (named Form CSV), Tafamidis glutaric acid co-crystal (named Form CSVI) and Tafamidis adipic acid co-crystal (named Form CSVII). It further discloses pharmaceutical composition comprising a therapeutically effective amount of Form CSV, Form CSVI or Form CSVII and pharmaceutically acceptable carriers or excipients such as dibutylhydroxytoluene and polysorbate.

U.S. Pat. No. 11,878,081 discloses pharmaceutical unit-dose formulations suitable for oral administration that contain from 1% w/w to 20% w/w of a tafamidis-organic acid (such as adipic acid, glutaric acid, or fumaric acid) co-crystal(s), organic acid dissolution enhancer, polyethylene glycol, and polysorbate.

U.S. Pat. Pub. No. 2023/0149366 discloses a solid oral dosage form containing tafamidis or a pharmaceutically acceptable salt or its solid-state forms or polymorphic forms thereof; at least one acidifier in an amount of about 0% w/w to about 10% w/w based on the total weight of the dosage form at least one pharmaceutically acceptable excipient. It further emphasizes the use of granulation and spray drying or evaporating methods.

WO 2024/023710 discloses tablet formulations containing Tafamidis free acid, one or more diluents, disintegrant and a lubricant. It emphasizes the use of disintegrant, such as cross-povidone, in higher amounts of 7-9% as compared to typical 1-5% to achieve the desired optimal release characteristic as compared with the RLD.

Hence there is a need to develop alternative tablet formulations of Tafamidis, Tafamidis meglumine or Tafamidis co-crystal(s), which are simple and easy to manufacture, cost effective and commercially viable at large scale. It is also desirable to develop tablet formulations of Tafamidis that do not form rigid gels upon contacting with water or buffer solution, and also do not require spray-dried or evaporation methods and/or the use of acidifiers. It is a further objective to develop tablet formulations of Tafamidis that achieve suitable dissolution profiles upon storage. Yes further, it is an objective to develop a tablet formulation that is smaller in size for ease of administration and to ensure better patient compliance.

The present novel tablet formulations fulfill the above-mentioned objectives.

### SUMMARY OF THE INVENTION

In one embodiment of the present invention, a pharmaceutically acceptable tablet formulation includes:
an effective amount of tafamidis or a pharmaceutically acceptable salt(s) thereof or tafamidis co-crystal(s); and
an effective amount of water-soluble excipient(s).

In some embodiments, the water-soluble excipients are selected from cyclodextrin or its derivatives, inorganic ionic compounds, carbohydrates, or a mixture thereof in the range of about 30% to about 99.5%.

In some embodiments, the tablet formulation of tafamidis or a pharmaceutically acceptable salt(s) thereof or tafamidis co-crystal(s) further contains at least one excipient selected from a diluent, a disintegrant and a lubricant.

In some embodiments, the tablet dosage forms include film coated tablets, dispersible tablets, orally disintegrating tablets, tablet in capsule, or powder for suspension.

In some embodiments, the tablet formulation is prepared by a dry blending process or wet granulation process.

In some embodiments, the tablet formulation is prepared by blending (physical mixture) of soluble excipients, including cyclodextrin or its derivatives, inorganic acids, carbohydrates and tafamidis or a pharmaceutically acceptable salt(s) thereof or tafamidis co-crystal(s), followed by blending with other excipients.

In some embodiments, the tablet is prepared by wet granulation process and further includes other excipients including, but not limited to, diluents, disintegrants, and lubricants.

In some embodiments, the tablet is prepared by grinding the water-soluble excipients and tafamidis or a pharmaceutically acceptable salt(s) thereof or tafamidis co-crystal(s), optionally followed by blending with other excipients.

In some embodiments, the tablet formulation is prepared by a standard manufacturing process.

The tablet pharmaceutical formulation of tafamidis or a pharmaceutically acceptable salt(s) thereof or tafamidis co-crystal(s) of the present invention provides a faster dissolution profile than the RLD.

In a further embodiment of the present invention, the tablet formulation of tafamidis or a pharmaceutically acceptable salt(s) thereof or tafamidis co-crystal(s) is prepared by following method:
The water-soluble excipients are granulated with a small quantity of solvent. The wet mass is dried and sized to obtain uniform granules.
tafamidis or a pharmaceutically acceptable salt(s) thereof or tafamidis co-crystal(s), and optionally, other excipients such as diluents, disintegrants, and lubricants are added to the granules, mixed, compressed as a tablet, and optionally coated.

In another embodiment of the present invention, the tablet pharmaceutical preparation of tafamidis or a pharmaceutically acceptable salt(s) thereof or tafamidis co-crystal(s) is used for the treatment of transthyretin amyloid disease and cardiomyopathy.

In some embodiments, a pharmaceutically acceptable tablet formulation includes an effective amount of at least one of tafamidis, a pharmaceutically acceptable salt thereof, and a tafamidis co-crystal; at least one solubilizing agent including at least one of cyclodextrin, substituted cyclodextrin derivatives, and mixtures thereof; and at least one excipient selected from diluent, disintegrant and lubricant.

In certain embodiments, the at least one solubilizing agent is present in an amount of about 10% wt. to about 99.5% wt.

In some embodiments, the pharmaceutically acceptable salt of tafamidis is tafamidis meglumine in a range from about 10 mg/tablet to about 100 mg/tablet. In certain of those embodiments, the effective amount of tafamidis meglumine is about 80 mg/tablet.

In some embodiments, the effective amount of tafamidis or the tafamidis co-crystal is about 12.2 mg/tablet. In other embodiments, the effective amount of tafamidis or the tafamidis co-crystal is about 61 mg/tablet.

In some embodiments, the tafamidis co-crystal is selected from fumaric acid, adipic acid, glutaric acid, and a mixture thereof. In certain of these embodiments, the tafamidis co-crystal is tafamidis fumaric acid.

In certain embodiments, the cyclodextrins, substituted cyclodextrin derivatives, and mixtures thereof are provided in a range from about 10 wt. % to about 60 wt. %.

In some embodiments, the formulation comprises the lubricant provided in a range from about 0.3 wt. % to about 5 wt. %.

The diluent may be provided in a range from about 10 wt. % to about 60 wt. %. The disintegrant may be provided in a range from about 1 wt. % to about 10 wt.

In certain embodiments, the formulation may include an outer film coating.

In some embodiments, the formulation exhibits at least 80% drug release in about 30 minutes.

In some embodiments, the at least one solubilizing agent is soluble in 900 ml of a dissolution medium. In additional embodiments, the at least one solubilizing agent may further include at least one of inorganic compounds, carbohydrates and mixtures thereof.

In certain embodiments, the tafamidis, the pharmaceutically acceptable salt thereof, and the tafamidis co-crystal has a D90 particle size of about 20 microns or less.

The present invention also provides a pharmaceutically acceptable tablet formulation, including an effective amount of at least one of tafamidis, a pharmaceutically acceptable salt thereof, and a tafamidis co-crystal, having a D90 particle size of about 20 microns or less; at least one solubilizing agent selected from cyclodextrin, substituted cyclodextrin derivatives, inorganic compounds, carbohydrates and mixtures thereof; and at least one excipient selected from diluent, disintegrant and lubricant; wherein the formulation is essentially free of acidifiers.

In further embodiments, the present invention provides a pharmaceutically acceptable tablet formulation, including at least one of tafamidis, a pharmaceutically acceptable salt thereof, and a tafamidis co-crystal, having a D90 particle size of about 20 microns or less; a cyclodextrin or a derivative thereof; an inorganic ionic compound; and a carbohydrate compound.

In yet further embodiments, a method of treating a transthyretin amyloid disease in mammals is provided, including administering a therapeutically effective amount of the pharmaceutically acceptable tablet formulation described above to a mammal in need thereof.

Other features and advantages of the present invention will become apparent from the following more detailed description, which illustrates, by way of example, the principle of the invention.

### DETAILED DESCRIPTION OF THE INVENTION

The terms “drug” or “active ingredient” refer to an agent, active ingredient compound or other substance, or compositions and mixture thereof that provide some pharmacological, often beneficial, effect. Reference to a specific active ingredient shall include where appropriate the active ingredient and its pharmaceutically acceptable salts.

The term “dosage form” refers to any form of the formulation, e.g., a tablet or capsule or parenteral, which comprises a pharmaceutically effective amount of an active ingredient sufficient to achieve a therapeutic effect and contains other excipients.

The term “co-crystal(s)” refers to a single-phase crystalline solid material composed of at least two different molecular and/or ionic compounds that are neither solvates nor simple salts and that exist in a stoichiometric ratio.

It is noted that, as used in the specification and the claims, the singular form “a,” “an,” and “the” comprises plural referents unless the context clearly indicates otherwise. For example, reference to a component in the singular is intended to comprise a plurality of components.

The term “about” is to be construed as modifying a term or value such that it is not an absolute. This term will be defined by the circumstances. This includes, at the very least, the degree of expected experimental error, technique error and instrument error for a given technique used to measure a value. In general, this term used in connection with a numerical value throughout the specification and the claims denotes an interval of accuracy, familiar and acceptable to a person skilled in the art. In general, such interval of accuracy is ±10%. Thus, “about ten” means 9 to 11. All numbers in this description indicating amounts, ratios of materials, physical properties of materials, and/or use are to be understood as modified by the word “about,” except as otherwise explicitly indicated.

The term “essentially free” means here less than 5 wt. %, preferably less than 3 wt. % and in particular less than 1 wt. %, based on the total weight of the composition.

The term “comprising” is synonymous with “including,” “having,” “containing,” or “characterized by.” These terms are inclusive and open-ended and do not exclude additional, unrecited elements or method steps.

The term “polymorph” refers to different crystalline forms of the same compound and includes, but is not limited to, other solid state molecular forms including hydrates (e.g., bound water present in the crystalline structure) and solvates (e.g., bound solvents other than water) of the same compound.

The term “stable” as used herein refers to physical stability, chemical stability, and polymorphic stability of a solid oral dosage form. Physical stability refers to consistent physical properties of composition throughout the product self-life, e.g., suspension/solution appearance, description, consistency, flow properties, manufacturability, and others. Chemical stability refers to consistency in obtaining acceptable results of drug assay, drug content uniformity and drug related substances. Polymorphic stability refers to retention of polymorphic type of Tafamidis API in the finished product and no polymorph conversions throughout the product self-life for e.g., at initial and at accelerated stability conditions (40° C./75% RH 6 months). The different physical properties exhibited by different solid forms of a pharmaceutical compound can affect important pharmaceutical parameters such as storage, stability, compressibility, density (important in formulation and product manufacturing), and dissolution rates (important in determining bioavailability). Stability differences may result from changes in chemical reactivity (e.g., differential hydrolysis or oxidation, such that a dosage form comprising a certain polymorph can discolor more rapidly than a dosage form comprising a different polymorph), mechanical changes (e.g., tablets can crumble on storage as a kinetically favored crystalline form converts to thermodynamically more stable crystalline form), or both (e.g., tablets of one polymorph can be more susceptible to breakdown at high humidity). Solubility differences between polymorphs may, in extreme situations, result in transitions to crystalline forms that lack potency and/or that are toxic. In addition, the physical properties of a crystalline form may also be important in pharmaceutical processing.

The term “water-soluble excipients” refers to compounds which are freely soluble in water. Some preferred water-soluble excipients include, but are not limited to, cyclodextrin and/or its derivatives, inorganic ionic compounds, and carbohydrate compounds. The water-soluble excipients may be dissolved in the 900 mL of dissolution medium.

The term “inorganic ionic compound” refers to a compound which has positive and negative ions, is soluble in water and increases wetting/channeling of a powder mixer in dry form or wet form.

The term “carbohydrate” refers to sugar compounds that are soluble in water and increase the channeling of the powder mixer in dry form or wet form.

The term “lubricant” refers to agents added to tablet and capsule formulations, typically in a small quantity (usually around 0.25%-5.0% wt.), to improve powder processing properties of formulations.

The term “diluent” refers to a substance that increases the size of a dosage form. Diluents are usually added to tablets or capsules where the active constituent is in low dose and to improve the powder flow and compaction properties prior to direct compression. Diluents are commonly used in a range between about 5% and about 80% wt.

The term “disintegrant” refers to the substance that is an additive that promotes disintegration, which is a breakage of a tablet into small fragments when in contact with a liquid medium.

The term “solid dosage form” refers to a solid pharmaceutical preparation in the form of, for example, a tablet, capsule, pill, powder, or granule.

The present invention provides a pharmaceutically acceptable solid oral formulation of Tafamidis or a pharmaceutically acceptable salt thereof or Tafamidis co-crystal(s). Particularly, the present invention relates to an economically feasible and commercially viable pharmaceutically acceptable tablet formulation of Tafamidis or a pharmaceutically acceptable salt thereof or Tafamidis co-crystal(s) prepared with water soluble excipients. More particularly, the present invention relates to a tablet formulation of Tafamidis or a pharmaceutically acceptable salt thereof or Tafamidis co-crystal(s), which involves use of standard dry blending ingredients in a standard manufacturing process to provide a similar or faster dissolution profile than the RLD. The present invention also relates to film coated tablets, dispersible tablets, orally disintegrating tablets, tablets in capsule, or powder for suspension.

The present inventors have discovered unexpectedly that a simple formulation using standard water-soluble ingredients provides a faster dissolution profile than the RLD. The developed tablet formulation is comparable in size or smaller than the soft gelatin capsule formulation of the RLD and thus, is easy for administration and improves patient compliance.

In one embodiment of the present invention, an oral pharmaceutically acceptable tablet formulation includes:
an effective amount of tafamidis or tafamidis meglumine or tafamidis co-crystal(s) in any of its polymorphic forms; and
an effective amount of one or more water-soluble excipients.

The inventive formulations may be in a form of tablets, film coated tablets, dispersible tablets, orally disintegrating tablets, tablet in capsule, or powder for suspensions and manufactured by standard manufacturing process. The inventive formulations surprisingly exhibit a similar or improved dissolution profile as compared to the available RLD soft gelatin capsule.

In some embodiments, the effective amount of tafamidis or tafamidis co-crystal(s) is in a range of about 10 mg/tablet to about 100 mg/tablet. In certain of those embodiments, the effective amount of tafamidis is about 61.0 mg/tablet. In some embodiments, the effective amount of tafamidis or tafamidis co-crystal(s) is about 12.2 mg/tablet.

In some embodiments, the effective amount of tafamidis meglumine is in a range of about 10 mg/tablet to about 100 mg/tablet. In certain embodiments, the effective amount of tafamidis meglumine is about 80.0 mg/tablet.

In some embodiments, the tafamidis co-crystal(s) may include, but are not limited to, fumaric acid, adipic acid, glutaric acid, or mixtures thereof. In one preferable embodiment, the tafamidis co-crystal is tafamidis fumaric acid co-crystal. Examples of Tafamidis co-crystals are described in U.S. Pat. Nos. 11,795,152 and 11,878,081, the disclosure of which is incorporated herein by reference.

In some embodiments, the suitable water-soluble excipients include cyclodextrin and/or its derivatives, inorganic ionic compounds, carbohydrates, or mixtures thereof. The one or more water-soluble excipients may be provided in a range of about 10% wt. to about 99.5% wt.

In some embodiments, the cyclodextrin and/or its derivatives include, but are not limited to, α-, β-, or γ-cyclodextrin, substituted cyclodextrin derivatives like hydroxypropyl β-cyclodextrin, sulfobutylether-β-cyclodextrin, and mixtures thereof.

The cyclodextrin or its derivatives may be provided in a range from about 10% wt. to about 70% wt., preferably from about 20% wt. to about 60% wt., and more preferably about 30% wt. to about 50% wt.

In some embodiments, the inorganic ionic compound may be selected from sodium chloride, calcium chloride, potassium chloride, sodium formaldehyde sulphoxylate, lithium chloride, or a mixture thereof.

The inorganic ionic compound may be provided in a range from about 10% wt. to about 60% wt., preferably from about 10% wt. to about 40% wt., and more preferably about 10% wt. to about 20% wt. In some embodiments, the formulation does not include any inorganic ionic compounds.

In some embodiments, the carbohydrate compound may be selected from sucrose, sorbitol, mannitol, or a mixture thereof.

The carbohydrate compound may be provided in a range from about 10% wt. to about 60% wt., preferably from about 20% wt. to about 50% wt., and more preferably about 30% wt. to about 50% wt.

In some embodiments, the tablet formulation of tafamidis meglumine further contains at least one additional excipient selected from a diluent, a disintegrant and a lubricant.

The diluent may be selected from lactose, pregelatinized starch, corn starch, microcrystalline cellulose, mannitol, dicalcium phosphate, or a mixture thereof. In some embodiments, the formulation may be essentially free of, or free of, microcrystalline cellulose.

In some embodiments, the diluent is provided in a range from about 10% wt. to about 60% wt., preferably from about 15% wt. to about 50% wt., and more preferably about 15% wt. to about 40% wt.

The disintegrant may be selected from sodium starch glycolate (SSG), crospovidone, croscarmellose sodium, or a mixture thereof. In some embodiments, the formulation may be essentially free of, or free of, crospovidone.

The disintegrant may be provided in a range from about 1% wt. to about 10% wt., preferably from about 1% wt. to about 5% wt.

In some embodiments, the lubricant is selected from sodium stearyl fumarate, talc, magnesium stearate, or a mixture thereof.

The lubricant may be provided in a range from about 0.3% wt. to about 5% wt., preferably from about 0.3% wt. to about 3% wt., and more preferably about 1% wt. to about 2% wt.

In some embodiments, the formulations disclosed herein may be free or essentially free of acidifiers, such as organic acids and inorganic acids.

In some embodiments, the tablet formulation of tafamidis optionally comprises coating, particularly the coating may comprise film coating, wherein the film coating material is but not limited to Opadry.

The inventive formulations may be prepared with a dry blending process or wet granulation process. In some embodiments, the inventive formulations are prepared by dry blending process only. In some embodiments, the manufacturing process for the inventive formulations does not involve evaporation or spray-drying steps. In some embodiments, the formulation may be essentially free of, or free of, water. In some embodiments, the process of manufacturing the inventive formulations does not involve the use of water or another solvent.

The inventive formulation surprisingly exhibits a similar or improved dissolution profile as compared to the available RLD soft gelatin capsule formulation.

The inventive formulations are prepared by simple manufacturing process. The inventive formulation retains the original polymorphic form of Tafamidis in the formulation. On the other hand, the soft gelatin capsule formulations of RLD are difficult to manufacture, are very variable in dissolution results, involve a high cost of production and are prone chemical degradation and polymorphic conversion.

### Experimental

General Manufacturing process for the preparation of a pharmaceutically acceptable tablet formulation of the present invention:
a) Raw material was dispensed as per batch composition.
b) Raw materials were sifted using a vibratory sifter.
c) Tafamidis meglumine, hydroxypropyl β-cyclodextrin (alone or in combination with sodium chloride and sorbitol), pregelatinised starch, sodium starch glycolate were mixed in high shear mixer granulator.
d) The material of step-c was granulated with separately prepared solution of povidone in IPA, in rapid mixer granulator until a suitable granular wet mass was obtained.
e) The obtained wet mass was dried, sized and mixed further with other excipients, such as sodium starch glycolate and colloidal silicon dioxide, in a double cone blender.
f) The material obtained in step-h was compressed on tablet press using suitable tooling to obtain tablets.
or
g) The material obtained in step-h was compressed on tablet press using suitable tooling to obtain tablets and coated using Opadry.

Alternatively, the following general manufacturing process may be used to prepare a pharmaceutically acceptable tablet formulation of the present invention:
h) Tafamidis meglumine, hydroxypropyl β-cyclodextrin, sodium chloride & sorbitol were directly mixed in double cone blender.
i) Sodium stearyl fumarate was separately sifted using a vibratory sifter.
j) The sifted sodium stearyl fumarate was added into the step-e or step-f in a double cone blender and mixed.
k) The material obtained in step-h was compressed on tablet press using suitable tooling to obtain tablets.
or
l) The material obtained in step-h was compressed on tablet press using suitable tooling to obtain tablets and coated using Opadry.

In some embodiments, the following general manufacturing process is used for the preparation of pharmaceutically acceptable tablet formulation of the present invention:
a) Raw material was dispensed as per batch composition.
b) Sorbitol was sifted using a vibratory sifter.
c) The material of step-b was loaded into a double cone blender.
d) Tafamidis, hydroxypropyl β-cyclodextrin alone, or hydroxypropyl β-cyclodextrin and sodium chloride, was co-sifted using a vibratory sifter. Optionally, other excipients are co-sifted and added.
e) The material of step-d was loaded into the step-c in a double cone blender and mixed.
or
f) The material of step-d was granulated with aqueous binder solution, dried, milled and mixed with extra-granular ingredients.
g) Sodium stearyl fumarate was separately sifted using a vibratory sifter.
h) The sifted sodium stearyl fumarate was added into the step-e in a double cone blender and mixed.
or
i) The material obtained in step-h was compressed on a tablet press using suitable tooling to obtain compressed tablets.
or
The material obtained in step-h was compressed on a tablet press using suitable tooling to obtain compressed tablets and coated using Opadry.

### Packaging of Pharmaceutically Acceptable Tablet Formulation of Tafamidis Meglumine of the Present Invention:

Compressed tablets were packaged in the below pack configurations:
10's Aluminum blister pack (3 blisters to be packed in one carton), and
30's HDPE bottle pack.

### EXAMPLES

### Example 1: Preparation of Pharmaceutically Acceptable Tablet of Tafamidis 61 mg

| Sr. No. | Ingredients | mg/tablet | % w/w |
|---|---|---|---|
| 1 | Tafamidis | 61.00 | 6.10 |
| 2 | Hydroxypropyl β-cyclodextrin | 360.00 | 36.00 |
| 3 | Sodium Chloride | 120.00 | 12.00 |
| 4 | Sorbitol | 448.00 | 44.80 |
| 5 | Sodium stearyl fumarate | 11.00 | 1.10 |

| | | |
|---|---|---|
| Tablet weight (mg) | 1000.00 | 100.00 |

### Example 2: Preparation of Pharmaceutically Acceptable Tablet of Tafamidis 61 mg

| Sr. No. | Ingredients | mg/tablet | % w/w |
|---|---|---|---|
| 1 | Tafamidis | 61.00 | 8.53 |
| 2 | Hydroxypropyl β-cyclodextrin | 294.50 | 41.19 |
| 3 | Sodium Chloride | 144.00 | 20.14 |
| 4 | Pregelatinised starch 1500 | 140.5 | 19.65 |
| 5 | Povidone K30 | 8.0 | 1.12 |
| 6 | Water | q.s | q.s |
| 7 | Crospovidone | 30.00 | 4.20 |
| 8 | Colloidal silicon dioxide | 14.00 | 1.96 |
| 9 | Sodium stearyl fumarate | 8.00 | 1.12 |

Coating

| | | | |
|---|---|---|---|
| 10 | Opadry beige | 15.00 | 2.10 |

| | | |
|---|---|---|
| Tablet weight (mg) | 715.00 | 100.00 |

In one embodiment, the inventive formulation may utilize a micronized form of Tafamidis (e.g., D90 being less than 20 microns).

In some embodiments, the present inventive formulation may contain any suitable polymorphic form(s) of Tafamidis or Tafamidis co-crystal(s) or Tafamidis solvate(s).

In some embodiments, the tablet formulation may contain hydroxypropyl β-cyclodextrin, sodium chloride and carbohydrate compound as key excipients, which are expected to provide an improved dissolution profile as compared with the RLD.

Dissolution results of the inventive tablet formulation of Tafamidis in comparison to the approved RLD soft gelatin capsule formulation are presented below in Table 1.

TABLE 1
Dissolution data of the inventive formulation of
Tafamidis 61 mg tablet per Example 1 and RLD:

| | Product | | | |
|---|---|---|---|---|
| | RLD-VYNDAMAX ® | | Inventive Formulation | |
| | Media | | | |
| | 0.05M phosphate buffer pH 6.8 with 1% tween 80, 900 ml, | | | |
| | Paddle with sinker, 75 RPM (temperature: 37 ± 0.5° C.) | | | |
| | | % Relative | | % Relative |
| | | Standard | | Standard |
| Time (min) | % Release | Deviation | % Release | Deviation |
| 15 | 7.6 | 103.5 | 84.1 | 1.8 |
| 30 | 86.1 | 5.8 | 91.8 | 1.8 |
| 45 | 94.3 | 1.3 | 92.5 | 4.0 |

TABLE 2
Dissolution data of the inventive formulation of
Tafamidis 61 mg tablet per the Example 2 and RLD:

| | Product | | | | | |
|---|---|---|---|---|---|---|
| | | | | | Inventive Formulation | |
| | | | | | (stored at accelerated | |
| | | | | | conditions 40° C. ± 2° C./75% | |
| | RLD-VYNDAMAX ® | | Inventive Formulation | | RH ± 5% RH, 3 Months) | |
| | Media | | | | | |
| | 0.05M phosphate buffer pH 6.8 with 1% tween 80, 900 ml, | | | | | |
| | Paddle with sinker, 75 RPM,_(temperature: 37 ± 0.5° C.) | | | | | |
| | | % Relative | | % Relative | | % Relative |
| Time | | Standard | | Standard | | Standard |
| (min) | % Release | Deviation | % Release | Deviation | % Release | Deviation |
| 15 | 7.6 | 103.5 | 66.3 | 10.0 | 60.4 | 15.1 |
| 30 | 86.1 | 5.8 | 96.0 | 1.7 | 93.1 | 1.8 |
| 45 | 94.3 | 1.3 | 97.5 | 1.3 | 96.1 | 0.9 |

### Example 3: Preparation of Pharmaceutically Acceptable Tablet of Tafamidis Meglumine 80 mg

| Sr. No. | Ingredients | mg/tablet | % w/w |
|---|---|---|---|
| 1 | Tafamidis meglumine | 80.00 | 7.84 |
| 2 | Hydroxypropyl β-cyclodextrin | 360.00 | 35.29 |
| 3 | Pregelatinized starch | 520.00 | 50.98 |
| 4 | Sodium starch glycolate (Type A) | 20.00 | 1.96 |
| 5 | Povidone K-30 | 20.00 | 1.96 |
| 6 | Isopropyl alcohol | q.s | — |
| 7 | Colloidal silicon dioxide | 8.00 | 0.78 |
| 8 | Sodium stearyl fumarate | 12.00 | 1.18 |

| | | |
|---|---|---|
| Tablet weight (mg) | 1020.00 | 100.00 |

### Example 4: Preparation of Pharmaceutically Acceptable Tablet of Tafamidis Meglumine 80 mg

| Sr. No. | Ingredients | mg/tablet | % w/w |
|---|---|---|---|
| 1 | Tafamidis meglumine | 80.00 | 8.00 |
| 2 | Hydroxypropyl β-cyclodextrin | 360.00 | 36.00 |
| 3 | Sodium Chloride | 120.00 | 12.00 |
| 4 | Sorbitol | 429.00 | 42.90 |
| 5 | Sodium stearyl fumarate | 12.00 | 1.20 |

| | | |
|---|---|---|
| Tablet weight (mg) | 1000.00 | 100.00 |

### Example 5: Preparation of Pharmaceutically Acceptable Tablet of Tafamidis Meglumine 80 mg

| Sr. No. | Ingredients | mg/tablet | % w/w |
|---|---|---|---|
| 1 | Tafamidis meglumine | 80.00 | 11.11 |
| 2 | Hydroxypropyl β-cyclodextrin | 360.00 | 50.00 |
| 3 | Pregelatinized starch | 207.00 | 28.75 |
| 4 | Sodium starch glycolate (Type A) | 30.00 | 4.17 |
| 5 | Povidone K-30 | 10.00 | 1.39 |
| 6 | Isopropyl alcohol | q.s. | — |
| 7 | Colloidal silicon dioxide | 5.00 | 0.69 |
| 8 | Sodium stearyl fumarate | 8.00 | 1.11 |
| | Coating | | |
| 9 | Opadry yellow | 20.00 | 2.78 |

| | | |
|---|---|---|
| Tablet weight (mg) | 720.00 | 100.00 |

In one embodiment, the inventive table formulation may utilize a micronized form of Tafamidis meglumine (e.g., D90 being less than 20 microns).

In some embodiments, the tablet formulation may contain Hydroxypropyl β-cyclodextrin and sodium chloride and carbohydrate compound used as key excipients, which is expected to provide improved dissolution. In additional embodiments, the formulation may not contain sodium chloride or another inorganic ionic compound.

Dissolution results of the inventive formulation of Tafamidis meglumine in comparison to the approved RLD soft gelatin capsule formulations are presented below in Table 3, Table 4 and Table 5.

TABLE 3
Dissolution data of pharmaceutically acceptable formulation of Tafamidis
meglumine 80 mg tablet prepared by present invention per Example 3:

| | Product | | | | | |
|---|---|---|---|---|---|---|
| | | | | | Inventive Formulation | |
| | | | | | (stored at accelerated | |
| | | | | | storage conditions 40° C. ± | |
| | | | | | 2° C./75% RH ± 5% RH, 3 | |
| | RLD-VYNDAQEL ® | | Inventive Formulation | | Months) | |
| | Media | | | | | |
| | 0.05M phosphate buffer pH 6.8, 900 ml, Paddle | | | | | |
| | with sinker, 75 RPM (temperature: 37 ± 0.5° C.) | | | | | |
| | | % Relative | | % Relative | | % Relative |
| Time | | Standard | | Standard | | Standard |
| (min) | % Release | Deviation | % Release | Deviation | % Release | Deviation |
| 15 | 24.5 | 52.4 | 96.0 | 2.2 | 85.7 | 0.7 |
| 30 | 85.2 | 2.7 | 103.7 | 0.5 | 95.8 | 0.4 |
| 45 | 91.9 | 4.3 | 104.0 | 0.4 | 95.7 | 0.4 |

TABLE 4
Dissolution data of pharmaceutically acceptable
formulation of Tafamidis meglumine 80 mg tablet
prepared by present invention per Example 4:

| | Product | | | |
|---|---|---|---|---|
| | RLD-VYNDAQEL ® | | Inventive Formulation | |
| | Media | | | |
| | 0.05M phosphate buffer pH 6.8, 900 ml, Paddle with | | | |
| | sinker, 75 RPM (temperature: 37 ± 0.5° C.) | | | |
| | | % Relative | | % Relative |
| | | Standard | | Standard |
| Time (min) | % Release | Deviation | % Release | Deviation |
| 15 | 24.5 | 52.4 | 36.6 | 8.0 |
| 30 | 85.2 | 2.7 | 44.2 | 7.5 |
| 45 | 91.9 | 4.3 | 88.2 | 6.4 |

TABLE 5
Dissolution data of pharmaceutically acceptable
formulation of Tafamidis meglumine 80 mg tablet
prepared by present invention per Example 5:

| | Product | | | |
|---|---|---|---|---|
| | RLD-VYNDAQEL ® | | Inventive Formulation | |
| | Media | | | |
| | 0.05M phosphate buffer pH 6.8, 900 ml, Paddle with | | | |
| | sinker, 75 RPM (temperature: 37 ± 0.5° C.) | | | |
| | | % Relative | | % Relative |
| | | Standard | | Standard |
| Time (min) | % Release | Deviation | % Release | Deviation |
| 15 | 24.5 | 52.4 | 80 | 1.9 |
| 30 | 85.2 | 2.7 | 95.4 | 0.2 |
| 45 | 91.9 | 4.3 | 95.5 | 0.4 |

It should be noted that the invention in its broader aspects is not limited to the specific details, representative compositions, methods, and processes, and illustrative examples described in connection with the preferred embodiments and preferred methods. Modifications and equivalents will be apparent to practitioners skilled in this art and are encompassed within the spirit and scope of the appended claims.

What is claimed is:

1. A pharmaceutically acceptable tablet formulation comprising;
an effective amount of at least one of tafamidis, a pharmaceutically acceptable salt thereof, and a tafamidis co-crystal;
at least one solubilizing agent selected from cyclodextrin, substituted cyclodextrin derivatives, and mixtures thereof; and
at least one excipient selected from diluent, disintegrant and lubricant.

2. The pharmaceutically acceptable tablet formulation according to claim 1, wherein the at least one solubilizing agent is present in an amount of about 10% wt. to about 99.5% wt.

3. The pharmaceutically acceptable tablet formulation according to claim 1, wherein the pharmaceutically acceptable salt of tafamidis is tafamidis meglumine in a range from about 10 mg/tablet to about 100 mg/tablet.

4. The pharmaceutically acceptable tablet formulation according to claim 3, wherein the effective amount of tafamidis meglumine is about 80 mg/tablet.

5. The pharmaceutically acceptable tablet formulation according to claim 1, wherein the effective amount of tafamidis or the tafamidis co-crystal is about 12.2 mg/tablet.

6. The pharmaceutically acceptable tablet formulation according to claim 1, wherein the effective amount of tafamidis or the tafamidis co-crystal is about 61 mg/tablet.

7. The pharmaceutically acceptable tablet formulation according to claim 1, wherein the tafamidis co-crystal is selected from fumaric acid, adipic acid, glutaric acid, and a mixture thereof.

8. The pharmaceutically acceptable tablet formulation according to claim 7, wherein the tafamidis co-crystal is tafamidis fumaric acid.

9. The pharmaceutically acceptable tablet formulation according to claim 1, wherein the cyclodextrins, substituted cyclodextrin derivatives, and mixtures thereof are provided in a range from about 10 wt. % to about 60 wt. %.

10. The pharmaceutically acceptable tablet formulation according to claim 1, wherein the formulation comprises the lubricant in a range from about 0.3 wt. % to about 5 wt.

11. The pharmaceutically acceptable tablet formulation according to claim 1, wherein the formulation comprises the diluent in a range from about 10 wt. % to about 60 wt. %.

12. The pharmaceutically acceptable tablet formulation according to claim 1, wherein the formulation comprises the disintegrant in a range from about 1 wt. % to about 10 wt.

13. The pharmaceutically acceptable tablet formulation according to claim 1, wherein the formulation comprises an outer film coating.

14. The pharmaceutically acceptable tablet formulation according to claim 1, wherein the formulation exhibits at least 80% drug release in about 30 minutes.

15. The pharmaceutically acceptable tablet formulation according to claim 1, wherein the at least one solubilizing agent is soluble in 900 ml of a dissolution medium.

16. The pharmaceutically acceptable tablet formulation according to claim 1, wherein the at least one solubilizing agent further comprises at least one of inorganic compounds, carbohydrates and mixtures thereof.

17. The pharmaceutically acceptable tablet formulation according to claim 1, wherein the tafamidis, the pharmaceutically acceptable salt thereof, and the tafamidis co-crystal has a D90 particle size of about 20 microns or less.

18. A pharmaceutically acceptable tablet formulation, comprising:
an effective amount of at least one of tafamidis, a pharmaceutically acceptable salt thereof, and a tafamidis co-crystal, having a D90 particle size of about 20 microns or less;
at least one solubilizing agent selected from cyclodextrin, substituted cyclodextrin derivatives, inorganic compounds, carbohydrates and mixtures thereof; and
at least one excipient selected from diluent, disintegrant and lubricant;
wherein the formulation is essentially free of acidifiers.

19. A pharmaceutically acceptable tablet formulation, comprising:
at least one of tafamidis, a pharmaceutically acceptable salt thereof, and a tafamidis co-crystal, having a D90 particle size of about 20 microns or less;
a cyclodextrin or a derivative thereof;
an inorganic ionic compound; and
a carbohydrate compound.

20. A method of treating a transthyretin amyloid disease in mammals, comprising administering a therapeutically effective amount of the pharmaceutically acceptable tablet formulation of claim 1 to a mammal in need thereof.
